SEQUENCE LISTING (16 sequences)

SEQ ID NO: 1 - protein, 691 aa, Pseudomonas aeruginosa
MATNTPKYGGLLTDIGAAALATASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLLGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKEADGELPGGVNLDSMVTSGWWSQSFTAQAASGANYPIVRAGLLHVYAASSNFIYQTYQAYDGESFYFRCRHSNTWFPWRRMWHGGDFNPSDYLLKSGFYWNALPGKPATFPPSAHNHDVGQLTSGILPLARGGVGSNTAAGARSTIGAGVPATASLGASGWWRDNDTGLIRQWGQVTCPADADASITFPIPFPTLCLGGYANQTSAFHPGTDASTGFRGATTTTAVIRNGYFAQAVLSWEAFGR

SEQ ID NO: 2 - protein, 691 aa, Pseudomonas aeruginosa
MTTNTPKYGGLLTDIGAAALAAASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLVGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKEADGELPGGVNLDSMVTSGWWSQSFTAQAATGANYPIVRAGLLHVYAASSNFIYQTYQAYDGESFYFRCRHSNTWFPWRRMWHGGDFNPSDYLLKSGFYWNALPGKPATFPPSAHNHDVGQLTSGILPLARGGVGSNTAAGARSTIGAGVPATASLGASGWWRDNDTGLIRQWGQVTCPADADASITFPIPFPTLCLGGYANQTSAFHPGTDASTGFRGATTTTAVIRNGYFAQAVLSWEAFGR

SEQ ID NO: 3 - protein, 702 aa, Pseudomonas aeruginosa
MTTNTPKYGGLLTDIGAAALAAASAAGKKWQPTHMLIGDAGGAPGATPDPIPAATQTKLINQRYRAQLNRLFVSDKNINTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLVGGGDLSADRSIGLAPSGVTAGSYRSVTVNANGVVTQGSNPSTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSTTSDPNTATVPLMLTNHANGPVAGRYFYIQSMFYPDQNGNASQIATSYNATSEMYVRVSYAANPSARDWLPWKRCDIGGSFSKEADGALGGAVNLNSLITSGWWYQTANAQAESGANYPVPRAGLLQVHNAGTNFIYQTYQVYDGEGFYFRCRYTNTWYPWRRVWHGADFNPNDYLLKSGFTWAALPGKPATFPPTGHNHDAAQITSGILPLARGGLGSNTAAGARNNIGAGVPATANRSLNGWWKDNDTGLIVQWMTVSVGDHPGGIVNRSLTFPIAFPTTCLHVVPSVKELGRPATSASTVTLADVSVSTTGCVIVATEYHGAVQNYAIRLVAIGC

SEQ ID NO: 4 - protein, 701 aa, Pseudomonas aeruginosa
MTTNTPKYGGLLTDIGAAALAAASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLVGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKTTDGSIGNGVNINSFVNSGWWLQSTSEWAAGGANYPVGLAGLLIVYRAHADHIYQTYVTLNGSTYSRCCYAGSWRPWRQNWDDGNFDPASYLPKAGFTWAALPGKPATFPPSGHNHDTSQITSGILPLARGGLGANTAAGARNNIGAGVPATASRALNGWWKDNDTGLIVQWMQVNVGDHPGGIIDRTLTFPIAFPSACLHVVPTVKEVGRPATSASTVTVADVSVSNTGCVIVSSEYYGLAQNYGIRVMAIGY

SEQ ID NO: 5 - protein, 691 aa, Pseudomonas aeruginosa
  MISC_FEATURE (324)..(324): Other
MTTNTPKYGGLLTDIGAAALAAASAAGKKWQPTHMLIGDAGGAPGDTLDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLLGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVXAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKEADGELPGGVNLDSMVTSGWWSQSFTAQAASGANYPIARAGLLHVYAASSNFIYQTYQAYDGESFYFRCRYSNTWLPWRRMWHGGDFNPSDYLLKSGFYWNALPGKPATFPPSAHNHDVGQLTSGILPLARGGVGSNTAAGARSTIGAGVPATASLGASGWWRDNDTGLIRQWGQVTCPADADASITFPIPFPTLCLGGYANQTSAFQPGTDASTGFRGATTTTAVIRNGYFAQAVLSWEAFGR

SEQ ID NO: 6 - protein, 913 aa, Yersiniophage L-413c
MSTKFKTVITTAGAAKLAAATVPGGKKVNLSAMAVGDGNGKLPVPDAGQTKLVHEVWRHALNKVSVDNKNKNYIVAELVVPPEVGGFWMRELGLYDDAGTLIAVSNMAESYKPELAEGSGRAQTCRMVIILSNVASVELSIDASTVMATQDYVDDKIAEHEQSRRHPDATLTEKGFTQLSSATNSTSEKLAATPKAVKAANDNANSRLAKNQNGADIQDKSAFLDNIGVTSLTFMKHNGMIPTTDNLDSYGPEEKYLGTWSCPSQSTAKPESGYPEDKGNGVLEVFNAGRFHCTQRYTTRTGNIYIRMLDAEWNPASPTWSAWRVITSGTRPLSTSIDLNSLGGAEHLGIWRNSSTSIASFERHFPEDGSFGQGILEVFEGGLYGRMQRYTTRSGTMYIRGLTASWDAENPQWEDWIAVGYQSTGWTYSGDLDDLLKPGIYSVTKQATNAPVTDSKDLAVGSIVEVKKRCDIESYIQTYTTVSATDAYKNRTFQRTRASGEADWGEWAEVYNSKSLLTKLGVGGVTDRLSSLDWQTYDFVPGSMITVRLSDMTNIPDGMEWGVIDTNLINITVGPSEGGGVARSMQVWRSTSNKTNYRFFTVRLYGNPGERSFNIRRLPIIDEAQTWEAKQTFSAGLSGELSGNAATATKLKTARKINNVSFDGTSDINLTPKNIGAFASGKTGDTVANDKAVGWNWSSGAYNATTGGASTLILHFNIGEGSCPAAQFRVNYKNGGIFYRSARDGYGFEADWSEFYTTTRKPTAGDVGALSLSGGQLNGALGIGTSSDLGGNSIVLGDNDTGFKQNGDGNLDVYANSVHVMRFVSGSIQSNKTINITGRVNPSDYGNFDSRYVRDVRLGTRVVQTMQKGVMYEKSGHVITGLGIVGEVDGDDPAVFRPIQKYINGTWYNVAQV

SEQ ID NO: 7 - protein, 920 aa, Artificial Sequence
  Synthetic construct - modified hmw bacteriocin
MATNTPKYGGLLTDIGAAALATASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAEHEQSRRHPDATLTEKGFTQLSSATNSTSEKLAATPKAVKAANDNANSRLAKNQNGADIQDKSAFLDNIGVTSLTFMKHNGMIPTTDNLDSYGPEEKYLGTWSCPSQSTAKPESGYPEDKGNGVLEVFNAGRFHCTQRYTTRTGNIYIRMLDAEWNPASPTWSAWRVITSGTRPLSTSIDLNSLGGAEHLGIWRNSSTSIASFERHFPEDGSFGQGILEVFEGGLYGRMQRYTTRSGTMYIRGLTASWDAENPQWEDWIAVGYQSTGWTYSGDLDDLLKPGIYSVTKQATNAPVTDSKDLAVGSIVEVKKRCDIESYIQTYTTVSATDAYKNRTFQRTRASGEADWGEWAEVYNSKSLLTKLGVGGVTDRLSSLDWQTYDFVPGSMITVRLSDMTNIPDGMEWGVIDTNLINITVGPSEGGGVARSMQVWRSTSNKTNYRFFTVRLYGNPGERSFNIRRLPIIDEAQTWEAKQTFSAGLSGELSGNAATATKLKTARKINNVSFDGTSDINLTPKNIGAFASGKTGDTVANDKAVGWNWSSGAYNATTGGASTLILHFNIGEGSCPAAQFRVNYKNGGIFYRSARDGYGFEADWSEFYTTTRKPTAGDVGALSLSGGQLNGALGIGTSSDLGGNSIVLGDNDTGFKQNGDGNLDVYANSVHVMRFVSGSIQSNKTINITGRVNPSDYGNFDSRYVRDVRLGTRVVQTMQKGVMYEKSGHVITGLGIVGEVDGDDPAVFRPIQKYINGTWYNVAQV

SEQ ID NO: 8 - protein, 146 aa, Pseudomonas aeruginosa
MIFFHAATGGFYSKEIHGSRMPLEDEMHPLEDAEYQALLRAQSEGKRIVTDHTGRPICVDPPAPAKDILVQRERIWRDRQLQLTDGPLARHRDEQDLGKTTTLSQEQLRELTLYRAVLRDWPIAAEFPDLNARPEPPAWLQSLITP

SEQ ID NO: 9 - protein, 152 aa, Pseudomonas aeruginosa
MKGEYYFSPSQVAFYPASLREVYEYAGCWPVDGEWVSAELHEQLMNEQAAGRAISSDVNGNPVAIERPPLSRQQRSTHERRWRDSQLLATDGLVVRHRDQLETGKETTLLPVQYHELMSYRASLRDWPEEPLFPDSGGRPSVPDWLRRYVTP

SEQ ID NO: 10 - protein, 152 aa, Pseudomonas aeruginosa
MKGEYYFSPSQVAFYPASLREVYEHAGCWPVDGEWVSAELHEQLMNEQAAGRAISSDVNGNPVAIERPPLSRQQRSTHERRWRDSQLLATDGLVVRHRDQLETGKETTLLPVQYHELMSYRASLRDWPEEPLFPDSGGRPSVPDWLRRYVTP

SEQ ID NO: 11 - protein, 152 aa, Pseudomonas aeruginosa
  MISC_FEATURE (17)..(17): Other
MKGEYYFSPSQVAFYPXSLREVYEYAGCWPVDGEWVSAELHEQLMNEQAAGRAISSDVNGNPVAIERPPLSRQQRSAHERRWRDSQLLATDGLVVRHRDQLETGKETTLLPVQYHELMSYRASLRDWPEEPLFPDSGGRPSVPDWLRRYVTP

SEQ ID NO: 12 - protein, 146 aa, Pseudomonas aeruginosa
MIFFHAATGGFYSKDVHGDRMPIDARMYPLEEAEYLALLVAQSEGKQIVADAAGRPFCIDPPAPAEEVLAHRERIWRDRQLTLTDGPIARHRDELDLGKITTLNQAQLLELTLYRASLRDWPASAAFPDLGARPEPPLWLEPLITP

SEQ ID NO: 13 - protein, 175 aa, Bacteriophage P2
MQHLKNIKSGNPKTKEQYQLTKNFDVIWLWSEDGKNWYEEVKNFQPDTIKIVYDENNIIVAITRDASTLNPEGFSVVEVPDITSNRRADDSGKWMFKDGAVVKRIYTADEQQQQAESQKAALLSEAENVIQPLERAVRLNMATDEERARLESWERYSVLVSRVDPANPEWPEMPQ

SEQ ID NO: 14 - protein, 175 aa, Bacteriophage L-413c
MQHLKNIKSGNPKTKEQYQLTKNFDVIWLWSEDGKNWYEEVSNFQEDTIKIVYDENNIIVGITRDASTFNPEGFSVVEVPDITANRRADDSGKWMFKDGAVIKRIYTADEQQQQAESQKAALLSEAESVIQPLERAVRLNMATDEERSRLEAWERYSVLVSRVDPANPEWPEMPQ

SEQ ID NO: 15 - protein, 669 aa, Bacteriophage P2
MSIKFRTVITTAGAAKLAAATAPGRRKVGITTMAVGDGGGKLPVPDAGQTGLIHEVWRHALNKISQDKRNSNYIIAELVIPPEVGGFWMRELGLYDDAGTLIAVANMAESYKPALAEGSGRWQTCRMVIIVSSVASVELTIDTTTVMATQDYVDDKIAEHEQSRRHPDASLTAKGFTQLSSATNSTSETLAATPKAVKAAYDLANGKYTAQDATTARKGLVQLSSATNSTSETLAATPKAVKTVMDETNKKAPLNSPALTGTPTTPTARQGTNNTQIANTAFVMAAIAALVDSSPDALNTLNELAAALGNDPNFATTMTNALAGKQPKDATLTALAGLATAADRFPYFTGNDVASLATLTKVGRDILAKSTVAAVIEYLGLQETVNRAGNAVQKNGDTLSGGLTFENDSILAWIRNTDWAKIGFKNDADGDTDSYMWFETGDNGNEYFKWRSRQSTTTKDLMTLKWDALNILVNAVINGCFGVGTTNALGGSSIVLGDNDTGFKQNGDGILDVYANSQRVFRFQNGVAIAFKNIQAGDSKKFSLSSSNTSTKNITFNLWGASTRPVVAELGDEAGWHFYSQRNTDNSVIFAVNGQMQPSNWGNFDSRYVKDVRLGTRVVQLMARGGRYEKAGHTITGLRIIGEVDGDDEAIFRPIQKYINGTWYNVAQV

SEQ ID NO: 16 - protein, 183 aa, Bacteriophage T4
MKIYHYYFDTKEFYKEENYKPVKGLGLPAHSTIKKPLEPKEGYAVVFDERTQDWIYEEDHRGKRAWTFNKEEIFISDIGSPVGITFDEPGEFDIWTDDGWKEDETYKRVLIRNRKIEELYKEFQVLNNMIEASVANKKEKFYYKNLKRFFALLEKHEHLGGEFPSWPEKEQKPWYKRLFKHYV

(19) United States
(12) Patent Application Publication
(10) Pub. No.: US 2008/0286236 A1
(43) Pub. Date: Nov. 20, 2008

(54) INHIBITION OF YERSINIA PESTIS

(75) Inventors: Dana M. Gebhart, San Francisco, CA (US); Dean M. Scholl, South San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AvidBiotics Corporation, South San Francisco, CA (US)

(21) Appl. No.: 11/748,437

(22) Filed: May 14, 2007

(51) Int. Cl. A61K 35/74; A01N 63/02; A61P 31/04; G01N 33/569; C12Q 1/04; A01P 1/00

(52) U.S. Cl. 424/93.2; 424/780; 424/93.6; 435/34; 435/7.32

(57) ABSTRACT

The disclosure relates to the targeting of Y. pestis mediated by the binding activity of tail fibers from naturally occurring R-type pyocins from Pseudomonas aeruginosa. The targeting may be mediated by a macromolecular complex such as the pyocin itself, a high molecular weight (hmw) bacteriocin modified to have the tail fiber's binding activity, or a bacteriophage modified to have the tail fiber's binding activity. Compositions comprising such complexes are described. Also disclosed are methods for the use of a complex, such as to inhibit the growth of a Yersinia species like Y. pestis, by compromising the integrity of its cytoplasmic membrane are also described. Additional methods include use of the binding activity to identify Y. pestis.

### FIELD OF THE DISCLOSURE

The disclosure relates to the targeting of Y. pestis mediated by the binding activity of tail fibers from naturally occurring R-type pyocins from Pseudomonas aeruginosa. The targeting may be mediated by a macromolecular complex such as the pyocin itself, a high molecular weight (hmw) bacteriocin modified to have the tail fiber's binding activity, or a bacteriophage modified to have the tail fiber's binding activity. Compositions comprising such complexes are described. Also disclosed are methods for the use of a complex, such as to inhibit the growth of, or to compromise the integrity of, the cytoplasmic membrane of a Yersinia species like Y. pestis. Additional methods include use of the binding activity to identify Y. pestis.

### BACKGROUND OF THE DISCLOSURE

Y. pestis is a gram-negative bacillus that causes the disease known as plague. Plague pandemics have occurred over the history of mankind and have killed tens of millions of people. The “Black Plague” killed over one third of the population of Europe during the Middle Ages, and there are still major plague epidemics in the world today.

The most common form of Y. pestis infection is bubonic plague. This disease occurs when the Y. pestis bacteria are transferred from rats to fleas and the fleas bite humans, wherein the disgorged plague bacteria then infect humans. Because it has a gestation of two to six days, bubonic plague, if diagnosed quickly, can be effectively treated with antibiotics, including doxycycline, streptomycin, gentamicin and ciprofloxacin assuming the bacteria are sensitive to these traditional antibiotics. However, if not treated rapidly or if not sensitive to the administered antibiotic, the plague bacteria can multiply in the blood and lymphatic system to form septicemic plague and when the lungs are infected, cause pneumonic plague. Pneumonic plague is the highly contagious, end stage of infection and can rapidly result in septic shock and death. Because the consequences of pneumonic plague occur so rapidly, pneumonic plague can have fatality rates from 50-90% even if treated with antibiotics to which the bacteria are sensitive, according to the Centers for Disease Control and Prevention.

Weaponized Y. pestis that has been aerosolized can present a serious bioterrorism threat because inhalation of the aerosol bacteria can lead directly to pneumonic plague. Since pneumonic plague is highly contagious, it is easily passed from human to human and animal to human through natural aerosols. Importantly, if such bacteria are engineered to resist first line antibiotics, they can become a virtually unstoppable bioweapon causing death rapidly after exposure. This threat would be even further exacerbated were a weaponized plague organism not sensitive to detection by plaque formation by the single phage, ΦA1122, used in the U.S. for rapidly identifying Y. pestis.

Prophylaxis would be an effective countermeasure to weaponized plague, particularly for those front line individuals with the highest risk of exposure to the bacteria. However, no prophylaxis currently exists for plague. There is not a vaccine available to protect the general population from Y. pestis, and there is a great reluctance to deploy prophylactic antibiotics out of fear of horizontal spread of drug resistance.

Alternatives to antibiotics for treating Y. pestis have been reported by Anisimov and Amoako 2006, but other alternative strategies must be explored.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE DISCLOSURE

The disclosed subject matter relates to the targeting and killing of Y. pestis as mediated by the binding activity of tail fibers present in naturally occurring R-type pyocins from Pseudomonas aeruginosa. Thus the disclosure includes, and is based in part on, the use of these pyocins to bind and kill Y. pestis. Without being bound by theory, the mechanism is likely through a compromise of the integrity of the cytoplasmic membrane of the Y. pestis.

Pyocins are a form of high molecular weight (hmw) bacteriocins that resemble but are distinct from bacteriophage tails. See FIG. 1. This particular class of bacteriocins includes R-type pyocins, tail-like bacteriocins, and R-type bacteriocins. For ease of reference, the term “hmw bacteriocin” will be used herein to refer to the bacteriocins of the disclosure, including, but not limited to, R-type bacteriocins, F-type and R-type pyocins, monocins, enterocoliticins, and meningocins.

Natural HMW bacteriocins are typically thermolabile, trypsin resistant, and can be induced by agents, which activate the SOS system. For example, they also have been identified in many enterobacteria, Pseudomonas species, Rhizobium lupin, Bacillus species, Yersinia species, and Flavobacterium species.

So in a first aspect, the disclosure includes a method of preventing or inhibiting the growth of Y. pestis, optionally to the point of killing Y. pestis. The method may include contacting a Y. pestis cell with an R-type pyocin which binds Y. pestis. The extent of inhibition may result in a loss of membrane potential or detectable release of some intracellular contents from the cell. In some embodiments, the R-type pyocin has the binding specificity of an R2, R4, or R5 pyocin as determined by their respective tail fiber proteins, SEQ ID NO: 1, 2, or 3, respectively. In other embodiments, the contacting is with an isolated R-type pyocin, such one or more of the R2, R4 and R5 pyocins. Where a combination of pyocins is used, any two or all three of these pyocins may be used.

In additional embodiments, the method may be used in vivo or in vitro. In some embodiments, the contacting occurs in vivo, such as where Y. pestis is present in a human patient or an animal subject as non-limiting examples. In other embodiments, the contacting is in vitro. One non-limiting example is where Y. pestis is present on a surface of an inanimate object and said contacting decontaminates the surface. In further embodiments, the method may be used to deactivate a bioweapon comprising Y. pestis. The method may include contacting the bioweapon with an R-type pyocin as described herein, such as an R-type pyocin which compromises the integrity of the cytoplasmic membrane Y. pestis.

In methods disclosed herein, the contacting may further include exposing, or contacting, the Y. pestis with an inhibitory antimicrobial or antibiotic. Non-limiting examples include doxycycline, streptomycin, gentamicin, and ciprofloxacin. In some embodiments, the antimicrobial or antibiotic inhibits log phase growth or retains Y. pestis in stationary phase.

To aid in the practice of these methods, a second aspect of the disclosure includes a composition containing one or more R-type pyocins that prevents or inhibits Y. pestis growth, optionally by killing Y. pestis cells. A composition may include more than one anti-Y. pestis pyocin. In many embodiments, the R-type pyocin has the binding specificity of an R2, R4, or R5 pyocin. In some embodiments, the composition includes one or more additional agents desired for use in anti-Y. pestis therapy or prophylaxis. Non-limiting examples of an additional agent include an antimicrobial, a bacteriophage, an antibiotic, an anti-fungal agent, an analgesic, and an anti-inflammatory agent. Independent of the pyocin, the antimicrobial or antibiotic may inhibit growth or proliferation of Y. pestis. In other embodiments, the antimicrobial or antibiotic also kills Y. pestis, optionally via a binding specificity and/or mechanism distinct from that of a disclosed pyocin.

In some cases, a pyocin in a composition of the disclosure is optionally purified or isolated prior to combination with one or more other components to form the composition. In other cases, a pyocin is not isolated prior to combination with one or more additional components. A disclosed composition may comprise a carrier or excipient. The carrier or excipient is one that is suitable for use in combination with a multisubunit complex like an R-type pyocin or other hmw bacteriocin as described herein. In some embodiments, the carrier or excipient is pharmaceutically acceptable such that the composition may be used clinically or agriculturally. In other embodiments, the carrier or excipient is suitable for topical, pulmonary, gastrointestinal, or systemic administration, such as to a human or a non-human animal. In additional embodiments, the carrier or excipient is suitable for administration to a non-animal organism, such as a plant as a non-limiting example.

A third aspect of the disclosure includes other compositions of the disclosure containing a recombinant or modified high molecular weight (hmw) bacteriocin, such as a recombinant R-type pyocin, or a recombinant or modified bacteriophage, such as a yersinia phage (“yersinophage”). These compositions optionally contain a disclosed Y. pestis killing pyocin. Such compositions may also contain an antimicrobial or antibiotic as disclosed herein. Therefore, all combinations of pyocins, recombinant or modified bacteriocins, and recombinant or modified bacteriophage are provided by the disclosure.

A fourth aspect of the disclosure includes a recombinant or modified hmw bacteriocin, which is a macromolecular complex composed of multiple copies of a number of different polypeptide subunits and possesses one or more tail fibers, altered to have the tail fiber binding activity of an R2, R4 or R5 pyocin as described herein. In some embodiments, the bacteriocin is an R1 or R3 pyocin modified to bind and kill Y. pestis by virtue of the receptor binding domain (RBD) from a tail fiber of an R2, R4 or R5 pyocin. In other embodiments, the bacteriocin is an enterocoliticin, such as one produced by Y. enterocolitica, modified to bind and kill Y. pestis via the RBD from a tail fiber of an R2, R4 or R5 pyocin.

Each tail fiber contains an RBD which binds to, or interacts with, a receptor to form a binding pair. The RBD is the binding portion of a tail fiber that makes it the first member of the binding pair. Therefore, and in some embodiments, the RBD of the tail fiber protein of an R1 (SEQ ID NO: 4) or R3 (SEQ ID NO: 5) pyocin is altered via modification of the protein to result in the binding activity of an R2, R4 or R5 RBD of a modified tail fiber. The receptor on the surface of Y. pestis to which the RBD binds is the second member of the binding pair.

So the disclosure includes an hmw bacteriocin, such as an R-type or F-type pyocin, with a modified tail fiber protein with the binding activity of the RBD from R2, R4, or R5 pyocin. In some embodiments, the modified tail fiber protein has one or more changes in the amino acid sequence of the RBD relative to a naturally occurring hmw bacteriocin. Non-limiting examples of a change in amino acid sequence include substitution, insertion (addition), or deletion of one or more amino acids. Of course combinations of one or more substitutions, insertions (additions), and deletions may also be used.

These modified bacteriocins may be used in a method to prevent or inhibit the growth of Y. pestis, optionally to the point of killing Y. pestis cells. The method may include contacting a Y. pestis cell with the modified bacteriocin. The contacting may result in a loss of membrane potential or a detectable release of intracellular components from the Y. pestis cell.

In a related fifth aspect, the disclosure includes a bacteriophage modified to have the tail fiber binding activity of a Y. pestis killing R-type pyocin. The bacteriophage prior to modification may be a yersinophage, non-yersinophage, or prophage. In some cases, the phage is a myoviridiae family member, optionally selected from P2 phages, P2-like phages, T-even phages, pseudo-T-even phages, and VHML. The modified or recombinant bacteriophage may have the binding specificity as an R2, R4 or R5 pyocin. In many embodiments, the bacteriophage is engineered to express a modified tail fiber protein to produce a modified RBD. In most embodiments, the modified RBD is derived from an R2, R4 or R5 pyocin or from the tail fiber (SEQ ID NO: 6) of yersiniophage L-413c.

Like the situation with modified bacteriocins, a modified or recombinant phage of the disclosure may be used in a method to prevent or inhibit the growth of Y. pestis, optionally to the point of killing Y. pestis cells. The method may include contacting a Y. pestis cell with the modified or recombinant phage. The contacting may result in a loss of membrane potential or detectable release of cellular factors from a Y. pestis cell.

The anti-Y. pestis methods described herein include methods of inhibiting Y. pestis cell growth, or inducing Y. pestis cell death. Such methods may include contacting a Y. pestis cell or cells with an effective amount of an anti-Y. pestis agent described herein. In some cases, an effective amount may be equivalent to as few as one, on average, pyocin per bacterial cell. Of course higher amounts may also be used. In further embodiments, a disclosed method may be used to compromise the integrity of the cytoplasmic membrane of a Y. pestis cell. The compromise may result in the loss of membrane potential and/or loss of some cellular contents. As described herein, the disclosed methods may include in vivo application (or administration) of an anti-Y. pestis agent within or on a subject. Alternatively, the methods may comprise in vitro contacting.

Other methods of using the modified or recombinant phage include inoculating a subject to provide protection against Y. pestis. The method may include administering, to the subject, recombinant bacteria which express a modified or recombinant phage with the binding specificity as an R2, R4 or R5 pyocin. The production and so presence of the phage may be used to produce a protected state in the subject against infection or colonization by Y. pestis. In other embodiments, the recombinant bacteria express a modified or recombinant hmw bacteriocin as described herein to produce an analogous protected state.

For the practice of methods involving recombinant bacteriocins or phages, the disclosure includes nucleic acid sequences encoding a modified tail fiber protein, as well as vectors and/or (host) cells containing the coding sequences. The vectors and/or host cells may be used to express the coding sequences to produce modified tail fiber proteins which form tail fibers and are incorporated into a disclosed modified or engineered hmw bacteriocin or bacteriophage.

A sequence encoding a modified tail fiber protein may also be introduced into a bacterial cell which produces, or is capable of producing, an hmw bacteriocin in the presence of the modified tail fiber protein. Expression of the modified tail fiber protein results in the production of a modified hmw bacteriocin by the cell. If endogenous bacteriocin tail fiber protein sequence(s) is/are inactivated or removed, then only modified hmw bacteriocins will be produced. The transfected bacteria may be propagated to produce hmw bacteriocins that prevent or inhibit the growth of Y. pestis, optionally to the point of killing Y. pestis.

In a further aspect, the disclosure includes methods to identify Y. pestis, optionally in the presence of one or more Yersinia species selected from Y. enterocolitica, Y. fredericksenii, and Y. pseudotuberculosis. These methods are based in part on the discovery that the binding activity of the R2, R4 and R5 pyocins is specific for Y. pestis at least with respect to these other species. Therefore, the disclosure includes a method of detecting the presence of Y. pestis in a sample containing one or more Yersinia species selected from Y. enterocolitica, Y. fredericksenii, and Y. pseudotuberculosis, wherein the sample is optionally suspected of containing Y. pestis. The method may include contacting it with a detectably labeled R-type pyocin which binds Y. pestis to form a complex and detecting the complex as an indicator of the presence of Y. pestis.

The detection of the complex may be by any suitable methodology. In some embodiments, it may be antibody mediated. In other embodiments, it may be mediated by an electrical signal, a fluorescent molecule, a quantum dot, an enzyme such as horseradish peroxidase.

In other embodiments, the method may include contacting the sample with an R-type pyocin which disrupts the cell membrane of Y. pestis and detecting the release of one or more intercellular components from Y. pestis as an indicator of the presence of Y. pestis in the sample. In some cases, the intracellular component may be detected by use of an antibody that specifically binds the component.

Alternatively, the method may include contacting the sample with a modified bacteriophage compromising a tail fiber with an RBD derived from R2, R4, and/or R5 and subsequently detecting replicated phages by their plaques formed on a lawn of cultured, known Y. pestis.

The details of one or more embodiments of the disclosure are set forth in the accompanying drawings and the description below. Other features, objects, and advantages of the disclosed subject matter will be apparent from the drawings and detailed description, and from the claims.

### BRIEF DESCRIPTION OF THE DRAWINGS

FIG. 1 provides the electron micrograph of an R-type pyocin particle revealing 4 of the 6 tail fibers in Panel A and a schematic of the major components of an R-type pyocin particle in Panel B.

FIG. 2 provides the determination of the LD₉₀ inoculum for P. aeruginosa. Ten female CD-1 mice were infected intraperitoneally with different inocula sizes of strain 13s P. aeruginosa. If and when animals first appeared moribund, they were euthanized, and survivors were counted at 24 (broken line) and 48 (solid line) hours post infection. The results are from 3 independent experiments conducted over 3 months.

FIG. 3 shows the effective treatment window for IV administration of pyocin. Female CD-1 mice were infected with LD₉₀ inocula of strain 13s P. aeruginosa. At each of the indicated times after infection, 10 animals were treated once intraveneously (IV) with 3×10¹¹ pyocins in 0.1 ml. If and when animals first appeared moribund, they were euthanized, and survivors were counted at 24 (broken line) and 48 (solid line) hours post infection.

FIG. 4 shows the response of infected animals to different IV doses of pyocins. Female CD-1 mice were infected with LD₉₀ inocula of strain 13s P. aeruginosa. One hour after infection 10 animals were treated once intraveneously with each of the indicated doses of pyocin in 0.1 ml. If and when animals first appeared moribund, they were euthanized, and survivors were counted at 24 (broken line) and 48 (solid line) hours post infection.

FIG. 5 shows pyocins R2, R4 and R5 kill Y. pestis bacteria. Activities of the R1 through R5 complemented pyocins were assessed by spotting onto indicator strain Pseudomonas aeruginosa 13s, which is sensitive to all pyocin types, panel A, and onto indicator strain Y. pestis KIM, panels B and C. In each of the panels or images A-C, the columns of bactericidal spots of serially (5×) diluted pyocin preparations are indicated along the tops according to the R-type pyocins (A & B) or the producer bacteria strains (C). In panel C, the columns entitled A1122 is a yersiniophage of that name, RΔ is the pyocin preparation from PA01Δprf15, P. aeruginosa strain PA01 produces R2 pyocin, NIH-H strain produces R5 pyocin, and R2-P2 is the modified pyocin comprising an RBD from phage P2. The R5 pyocin preparation used in panels A and B had previously lost all activity, but in panel C, the natural R5 pyocin produced by strain NIH-H is shown to actively kill Y. pestis.

FIG. 6 provides spot assays of R2 RBD-deleted pyocins that have been complemented with the RBD of tail fibers from pyocins R1, R2, R3, R4, R5, from phage P2 or from phage L-413c. In each of the images A-D, the columns of bactericidal spots of serially (5×) diluted pyocin preparations are numbered along the tops according to the sources of the RBDs. Column 1 was the “pyocin preparation” from PA01Δprf15; columns 2, 3, 4, 5, 6, 7, and 8, were pyocins made with the RBDs derived from tail fiber genes of R1, R2, R3, R4, R5, from phage P2 and from phage L-413c, respectively, complementing in trans the deleted R2 prf15 in PA01Δprf15. The indicator bacteria are: A. Pseudomonas aeruginosa strain 13s, which is sensitive to all 5 natural pyocins; B. Pseudomonas aeruginosa strain 13s R2^{r} (resistant to R2, R3 and R4 but sensitive to R5 pyocins); C. E. coli C1a; and D. Yersinia pestis KIM.

FIG. 7 provides trans complementation of the PA01Δprf15 R2 pyocin structure with various R-type pyocin tail fibers, tail fiber fusions and chaperones. Activities of the R1 through R5 complemented pyocins were assessed by spotting onto indicator strain Pseudomonas aeruginosa 13s, which is sensitive to all pyocin types. The R2-P2 complemented pyocins were tested for activity using E. coli C as the indicator, and the R2-L413c complemented pyocin was tested on Yersinia pestis strain KIM.

The R2, R3, and R4 Prf15 tail fibers could be chaperoned by the endogenous Prf16 of the PA01Δprf15 R2 pyocin. R1 and R5 Prf15 tail fibers, which differ at the C-terminus compared to R2, required their own cognate Prf16 (each of which differs in sequence from the R2 counterpart). Both the R2-P2 and R2-L413c fusions, which contain the C-terminus (RBD) of the phage P2 and L413c tail fibers, respectively, require their cognate tail fiber assembly chaperones encoded by their respective G genes.

FIG. 8 provides the amino acid sequences for SEQ ID NOS:1-16, provided on pages 8A-8D.

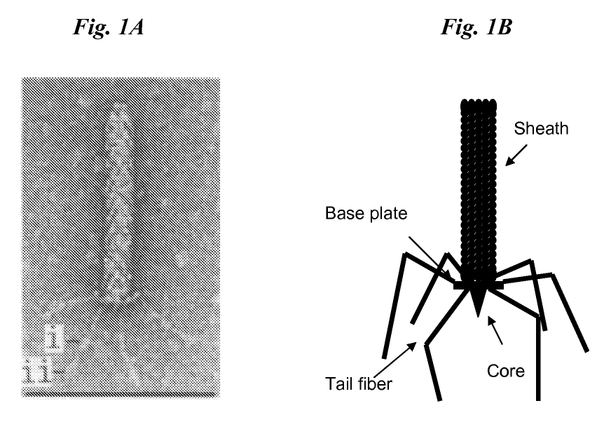

### DEFINITIONS

As used herein, an hmw bacteriocin includes an R-type pyocin, tail-like bacteriocin, R-type bacteriocin, F-type and R-type pyocins, monocins, meningocins, or other high molecular weight (hmw) bacteriocins. An hmw bacteriocin includes modified versions of R-type and F-type pyocins, enterocoliticins, monocins, and meningocins (see Kingsbury). A modified or engineered hmw bacteriocin may be a modified R-type pyocin selected from the R1, R2, R3, R4, or R5 pyocin of P. aeruginosa. A bacteriocin of the disclosure is generally mild acid resistant, trypsin resistant, sedimentable by centrifugation, and resolvable by electron microscope (see Jabrane; Daw et al.; and Kageyama et al. 1962). In many cases, an engineered hmw bacteriocin disclosed herein has one or more, in any combination, of these properties. An additional property common to bacteriocins and engineered hmw bacteriocins disclosed herein is that they are replication deficient such that they cannot reproduce themselves after binding to the surface of a target bacterium as can many bacteriophages.

Pyocins, and other hmw bacteriocins disclosed herein, are complex molecules comprising multiple protein, or polypeptide, subunits. In naturally occurring pyocins, the subunit structures are encoded by the bacterial genome, such as that of P. aeruginosa, and form pyocins to serve as natural defenses against other bacteria (Kageyama, 1975). A sensitive, target bacterium can be killed by a single pyocin molecule (Kageyama, 1964; Shinomiya & Shiga, 1979; Morse et al., 1980; Strauch et al., 2001).

The terms “inhibit growth” and “growth inhibition” or variations thereof refer to the slowing or stopping of the rate of a bacteria cell's division or cessation of bacterial cell division. The terms include the killing or death of the bacteria.

As used herein, a “nucleic acid” typically refers to deoxyribonucleotide or ribonucleotides polymers (pure or mixed) in single- or double-stranded form. The term may encompass nucleic acids containing nucleotide analogs or modified backbone residues or linkages, which are synthetic, naturally occurring, and non-naturally occurring, which have similar binding, structural, or functional properties as the reference nucleic acid, and which are metabolized in a manner similar to the reference nucleotides. Non-limiting examples of such analogs include, without limitation, phosphorothioates, phosphoramidates, methyl phosphonates, chiral-methyl phosphonates, 2-O-methyl ribonucleotides, and peptide-nucleic acids (PNAs). The term nucleic acid may, in some contexts, be used interchangeably with gene, cDNA, mRNA, oligonucleotide, and polynucleotide.

The terms “polypeptide,” “peptide,” and “protein” are typically used interchangeably herein to refer to a polymer of amino acid residues. Amino acids may be referred to herein by either their commonly known three letter symbols or by the one-letter symbols recommended by the IUPAC-IUB Biochemical Nomenclature Commission.

### DETAILED DESCRIPTION OF MODES OF PRACTICING THE DISCLOSURE

General

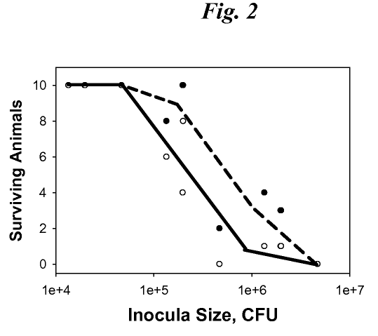

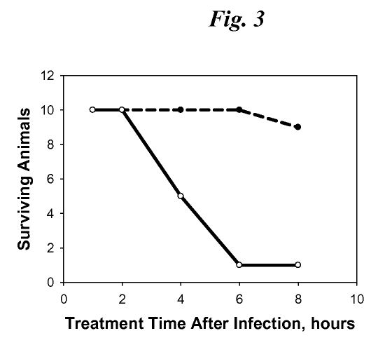

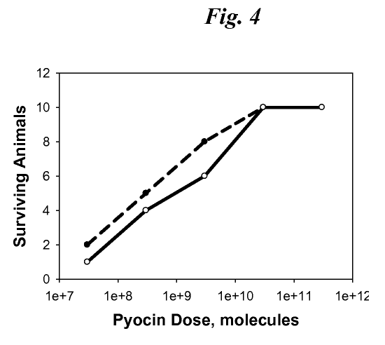

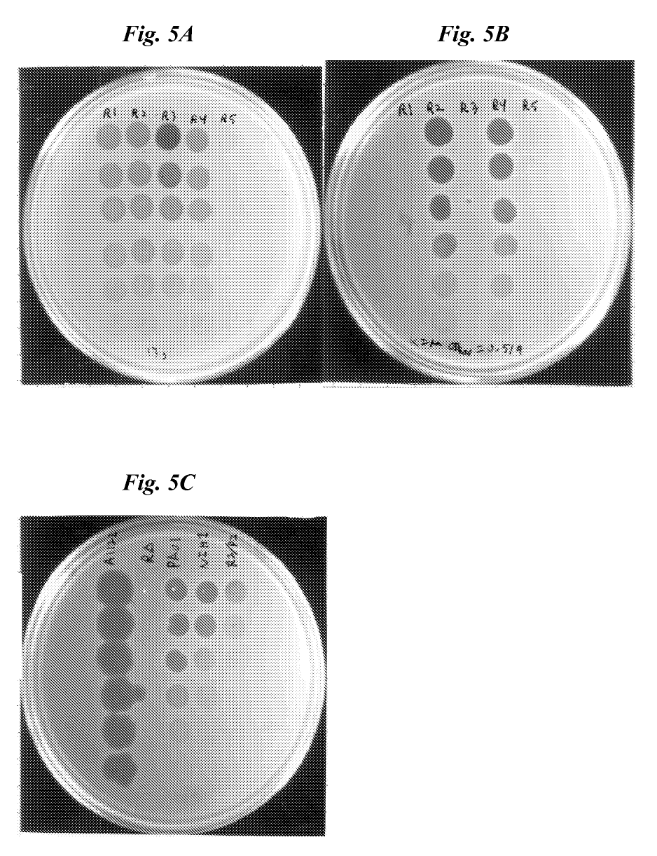

Pyocins are complex protein structures encoded by the genome of a bacterium, such as P. aeruginosa, and serve as natural defenses against other bacteria (Kageyama, 1975). A sensitive bacterium can be killed by a single pyocin molecule (Morse et al., 1980; Birmingham & Pattee, 1981; Strauch et al., 2001). Francois Jacob discovered and first described pyocins as high molecular weight (hmw) bacteriocins (Jacob, 1954). Although the word pyocin is commonly used to describe the hmw bacteriocins of P. aeruginosa, similar entities have been described in multiple other gram-negative bacteria (Coetzee et al., 1968) and even for Listeria moncytogenes (Zink et al. 1995) and Staphylococcus aureus (Thompson and Pattee, 1981), both gram-positive organisms. While pyocins morphologically resemble the tails of contractile (myoviridae) bacteriophages, they are not simply defective phages. There are meaningful differences, for example, in physical and chemical stability between pyocins and phage tails (Kageyama & Egami, 1962; Nakayama et al., 2000). The antibacterial efficacy of pyocins have demonstrated in mouse models of lethal bacterial infections (Merrikin & Terry, 1972; Haas et al., 1974); and as described below in FIGS. 2-4.

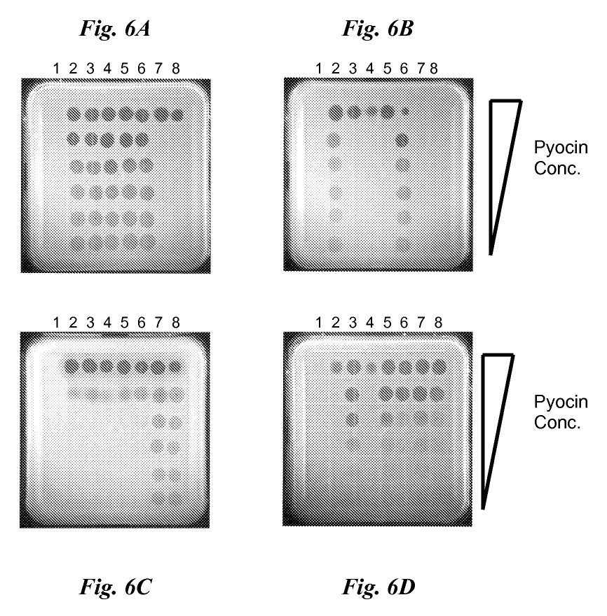

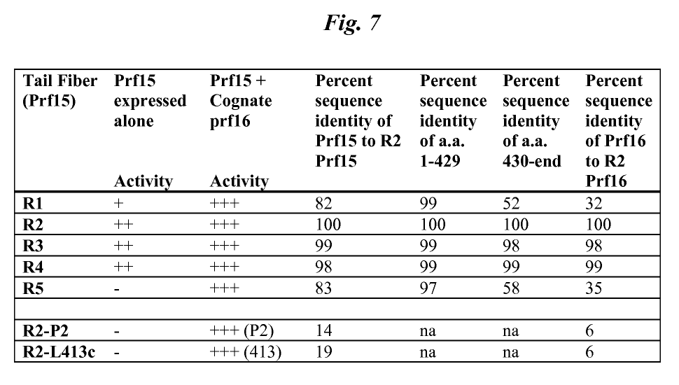

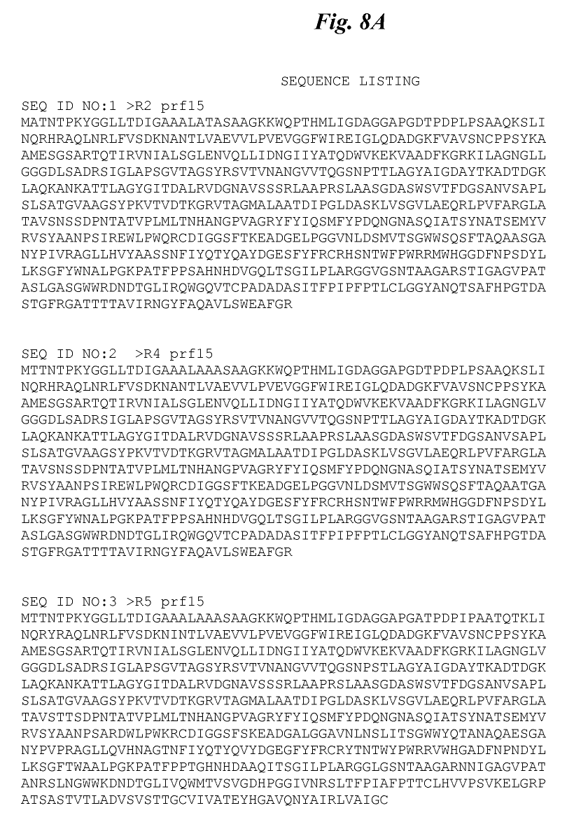

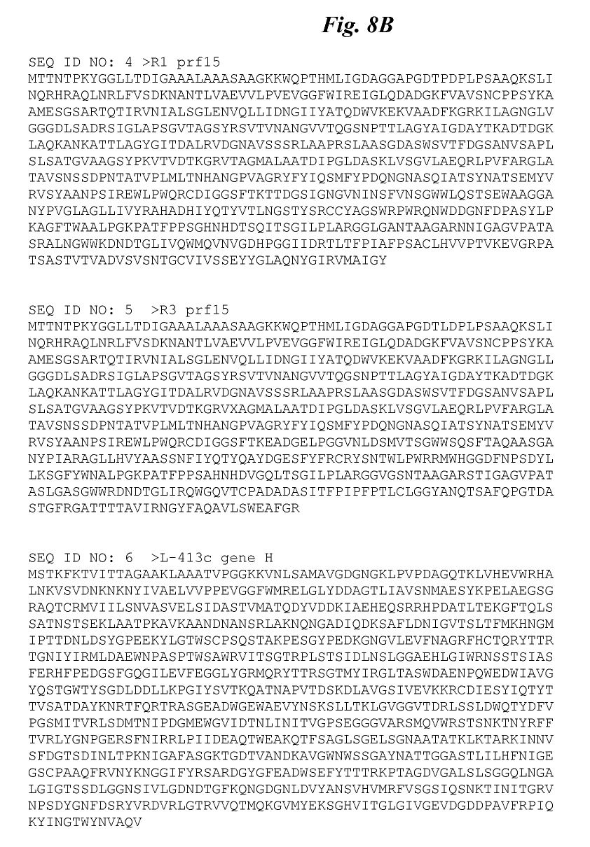

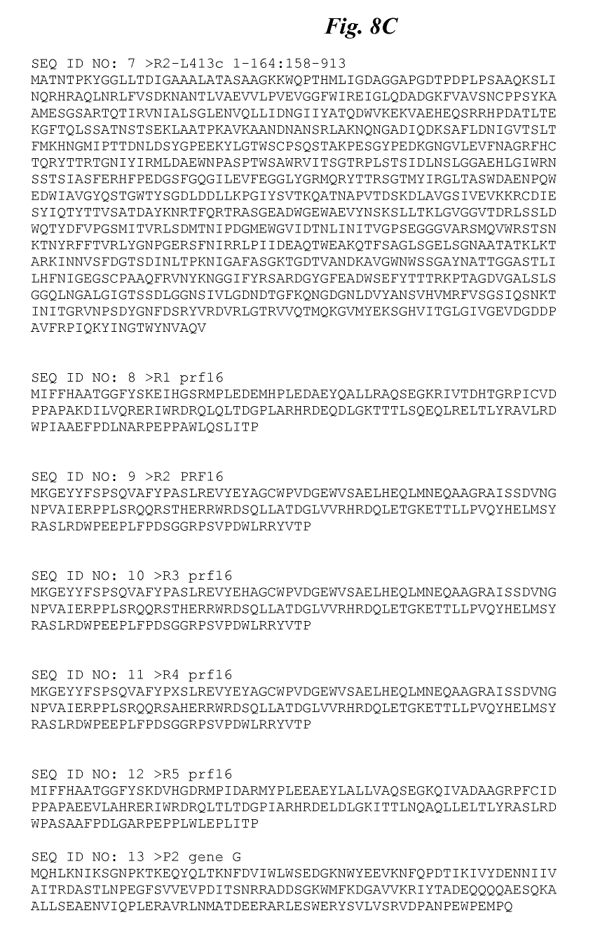

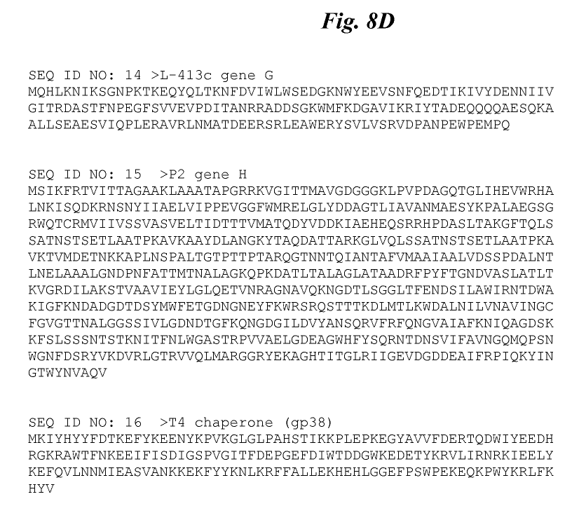

Certain R-type pyocins, specifically the natural (see FIG. 5) and recombinant (see FIG. 6) R2, R4 and R5 pyocins of Pseudomonas aeruginosa, effectively kill Yersinia pestis. See FIG. 5. The observed killing was with single-hit kinetics, such that as few as one pyocin can kill one Y. pestis bacterium. The utility of this discovery is enormous for protecting and treating humans and other animals with plague, particularly pneumonic plague. Delivering one or a cocktail of appropriately formulated and aerosolized bactericidal pyocins to the lungs of a person or animal exposed to aerosolized Y. pestis or having contracted pneumonic plague has the potential of being life-saving. Even antibiotic-resistant and/or phage-resistant plague might be treated effectively with one or more of the select pyocins. Additionally, inanimate surfaces may be decontaminated by the application of one or more of the select pyocins.

Pyocin-based therapeutics usually contain no genetic material and thus cannot replicate; can be dosed in a linear fashion, not as an exponentially expanding therapy; can target specifically and kill generically; and can by-pass numerous mechanisms that convey resistance to phage killing.

Binding Specificity of Anti-Y. pestis R-Type Pyocins

The disclosure includes the use of R-type pyocins in methods to prevent or inhibit their growth, optionally with inclusion of Y. pestis cellular toxicity. The methods may comprise contacting Y. pestis with an isolated R-type pyocin which binds thereto. The binding is mediated by the tail fiber of the pyocin, which resembles a bacteriophage tail fiber. A pyocin tail fiber includes a binding site, or receptor binding domain (RBD), as described herein that facilitates binding between the pyocin and Y. pestis. The binding mediates the pyocin's toxicity against Y. pestis. Thus a method of the disclosure may be practiced with use of any natural or modified R-type pyocin with the binding activity, or RBD, of an R2, R4, or R5 pyocin.

In some cases, the pyocin is a modified or recombinant hmw bacteriocin which has been changed relative to an unmodified, naturally occurring, or native bacteriocin by substitution of the native, or endogenous RBD, with the RBD from an R2, R4, or R5 pyocin. The term “recombinant”, typically used with reference to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. A recombinant cell expresses genes that are not found within the native (non-recombinant) form of the cell or expresses native genes that are abnormally expressed, under expressed, or not expressed at all. In some embodiments, the R2, R4, or R5 pyocin RBD may be substituted for the RBD of another pyocin, such as the R1 or R3 pyocin.

Pyocins and recombinant hmw bacteriocins are favored in the practice of the disclosure because many of them can be lyophilized and stored for significant periods of time. Lyophilized proteins may be aerosolized as solid micro-particles and inhaled to access the deepest lung (alveolar) spaces. Thus, aerosolized, lyophilized pyocins and bacteriocins may serve as a potential prophylaxis or treatment of weaponized plague for those individuals at high risk of exposure to the bioweapon. In alternative embodiments, a lyophilized material may be subsequently reconstituted and used in a method according to the disclosure.

In some embodiments, a modified or recombinant hmw bacteriocin with the binding specificity of an anti-Y. pestis R-type pyocin as described herein is prepared by introduction of the RBD from an R2, R4 or R5 pyocin into the bacteriocin. In many cases, the insertion is made with the deletion of the endogenous RBD from the bacteriocin. As a non-limiting example, the RBD from an R1 or R3 pyocin, or from enterocoliticin, may be substituted by an RBD from R2, R4 or R5 pyocin. As exemplified in FIG. 6, the RBD from an R2 pyocin can be substituted by the RBD of an R5 pyocin, and the resulting modified hmw bacteriocin then exhibits the killing spectrum not of an R2 pyocin but of an R5 pyocin, including the killing of Y. pestis.

This aspect of the disclosure is based on the properties of a disclosed pyocin to bind to, or interact with, a Y. pestis surface receptor to form a binding pair. The binding or interaction occurs through the RBD of the pyocin's tail fiber, which is the first member of the binding pair, with the receptor being the second member of the pair. In many embodiments, the receptor is a Y. pestis surface molecule.

A modified or engineered hmw bacteriocin disclosed herein comprises a tail fiber having both a base plate attachment region (BPAR) and a modified, or heterologous, RBD. The tail fiber is a trimeric structure of three tail fiber protein subunits, each of which also comprises a first domain corresponding to, and forming, the BPAR in a tail fiber and a second domain corresponding to, and forming, a modified or heterologous RBD in a tail fiber.

Typically, “heterologous” when used with reference to portions of a protein or nucleic acid sequence indicates that the sequence comprises two or more subsequences that are not normally found in the same relationship to each other in nature. For instance, a heterologous protein indicates that the protein comprises two or more subsequences that are not found in the same relationship to each other in nature. “Heterologous” also means that the amino acid or nucleic acid sequence is not normally found in conjunction with the other sequences or is not normally contained in the selected plasmid, vector, or host. In other words, it is not native to the system for which it is now utilized. For example, proteins produced by an organism that is not the wild type source of those proteins.

So in some embodiments, the disclosure includes an hmw bacteriocin tail fiber protein comprising a BPAR of the protein and a modified, or heterologous, RBD sequence. The BPAR is typically at the N-terminal region of a tail fiber protein, while the RBD is typically at the C-terminal region. An example of an hmw bacteriocin tail fiber protein comprising a BPAR of one tail fiber protein and a heterologous RBD sequence of another tail fiber is that created (SEQ ID NO: 7) by fusing R2 PRF15 (SEQ ID NO: 1) and phage L-413c tail fiber protein (SEQ ID NO: 6) encoded by gene H. Other than the modified, or heterologous, RBD, the tail fiber protein may be that of any naturally occurring hmw bacteriocin, with a pyocin, monocin, enterocoliticin, or meningocin being non-limiting examples. In some embodiments, the tail fiber protein sequences of R2, R4, and R5 pyocins, as represented by SEQ ID NOs:1, 2, and 3, respectively, may be used as described herein.

Embodiments of the disclosure include a combination with the N-terminal amino acids from position 1 to about position 164 or position 240 of a bacteriocin tail fiber protein. This polypeptide fragment may be fused to a region of an R2, R4 or R5 pyocin tail fiber protein including its C-terminus containing BPD. The region may be a polypeptide fragment lacking the N-terminal region from position 1 to about position 150, about position 164, about position 170, about position 190, about position 240, about position 290, about position 300, or about position 320. The fusion protein may be readily prepared by recombinant DNA techniques with nucleic acid sequences encoding an hmw bacteriocin tail fiber protein and the R2, R4 or R5 pyocin. One exemplary tail fiber protein coding sequence is R2 prf15, which encodes the R2 tail fiber protein, SEQ ID NO: 1.

In embodiments comprising the substitution of RBD amino acid residues, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 22%, about 24%, about 26%, about 28%, about 30%, about 35%, about 40%, about 45%, or about 50%, or more, of the C-terminal in a tail fiber protein are substituted. In some embodiments, the substitutions are within about 245, about 260, about 275, or about 290, or more, residues from the C-terminus.

The nucleic acid molecules described herein may be used to express and prepare tail fiber proteins, especially modified or engineered proteins, by any means known to the skilled person. In some embodiments, the expression is via the use of a vector containing the nucleic acid molecule(s) operably linked to a promoter that can direct the expression of the encoded tail fiber protein.

In many embodiments, the expression may occur with expression of an accessory gene, such as a “chaperone” encoding sequence reported for various bacteriocins and bacteriophages. The presence of a chaperone facilitates assembly of an hmw bacteriocin of the disclosure often without becoming a part of the bacteriocin, as shown in FIG. 7. The chaperone may be the cognate, or corresponding, protein for the BPD used in an hmw bacteriocin of the disclosure. One non-limiting example of a chaperone is encoded by prf16 of R1 pyocin (SEQ ID NO: 8), and it corresponds to (or is the cognate chaperone for) the R1 pyocin tail fiber protein (SEQ ID NO: 4) encoded by the R1 prf15 gene. As shown in FIG. 7, the R1 chaperone supports well only the formation of R1 pyocin tails, while the R2 prf16 encoded chaperone (SEQ ID NO: 9) supports the formation of pyocins with R2, R3, and R4 derived RBDs. The sequences of the chaperones for R2, R3 and R4 (SEQ ID NO: 9, 10, and 11, respectively) are nearly identical. The chaperone for R5 pyocin tail fiber (SEQ ID NO: 12) is very different from the other chaperones and exclusively supports the formation of tail fibers with R5 derived RBDs. Other examples of tail fiber protein chaperones include gene G in the P2 (SEQ ID NO: 13) and L413c (SEQ ID NO: 14) bacteriophages, which chaperone specifically supports the respective tail fiber protein genes H (SEQ ID NO: 15 and SEQ ID NO: 6, respectively) in each phage. The gene G products are homologues to the phage T4 gp38 (SEQ ID NO: 16), which is known to be responsible for proper folding of the tail fiber into trimers (Burda, and Miller 1999; Qu et at., 2004; and Hashemolhosseini et al., 1994). These chaperones seem to be common among the known Myoviridae.

The use of a cognate chaperone is advantageous because a non-cognate chaperone may be insufficient to correctly fold a given tail fiber protein and/or assemble it into an hmw bacteriocin, as shown in FIG. 7. As a non-limiting example, the R2 prf16 gene product has been observed to be preferred to complement the folding of the R2 tail fiber. So where an R2 tail fiber protein RBD sequence is used in a modified or recombinant bacteriocin of the disclosure, co-expression of the cognate prf16 gene product may be advantageously used in combination. Without being bound by theory, and offered to improve the understanding of the present disclosure, it is believed that a chaperone may act specifically on the C-terminal portion of its cognate tail fiber protein and that the tail fibers and their chaperones have co-evolved. However, Qu et al. isolated a T4 gp37 mutant that suppresses the requirement for gp38. This mutant had in gp37 a duplication of a coiled-coil motif, which may itself play a role in folding. Therefore, it is further believed that a tail fiber protein may be designed to contain such a change so that it folds properly without the need to co-express a cognate chaperone.

Therefore, embodiments of the disclosure include a bacterial cell transfected with a nucleic acid molecule encoding a modified or engineered tail fiber protein, optionally co-expressed with a chaperone, as described herein. Expression of the nucleic acid molecule, optionally with an accessory (chaperone) protein as necessary, results in the production of modified or engineered tail fibers of the disclosure. Sequences encoding the tail fiber protein and chaperone may be contained within a single nucleic acid molecule, such as a plasmid or other vector, or by separate molecules. Where a single nucleic acid molecule is used, the sequences optionally may be under the control of the same regulatory sequence(s). Alternatively, the coding sequences may be under separate regulatory control.

In some embodiments, the bacterial cell is also capable of expressing the additional subunits to form an hmw bacteriocin comprising a modified or engineered tail fiber. In one group of embodiments, the endogenous tail fiber protein coding sequence of the bacterial cell is inactivated or deleted. Optionally, the other subunits may be encoded by sequences on a nucleic acid molecule, such as a plasmid or other vector, separate from that which contains a sequence encoding a tail fiber protein and/or chaperone. Thus the tail fiber protein and/or chaperone may be provided one or more nucleic acid molecules in trans relative to the other subunits.

The nucleic acids, vectors, and bacterial cells may be used in a method of producing a modified or engineered hmw bacteriocin as disclosed herein. Such a method may comprise culturing a bacterial cell containing nucleic acid molecules as described above under conditions resulting in the expression and production of the tail fiber and so hmw bacteriocin. In some embodiments of the disclosure the conditions are in vivo within an animal.

In one group of embodiments, a method of preparing an hmw bacteriocin comprises expressing the bacteriocin subunits, including the modified or engineered tail fiber protein, in a host bacterium, and harvesting the hmw bacteriocin from the bacterial culture. The host bacterium is a complementary host production bacterium that encodes and expresses the other subunits necessary for the production of the bacteriocin. The term “host bacterium” or “host bacteria” refers to a bacterium or bacteria used to produce an hmw bacteriocin disclosed herein. Host bacteria or bacterium may also be referred to as “host production bacterium” or “host production bacteria”. The “harvesting an hmw bacteriocin from a bacterial culture” generally comprises removing the bacteriocin from the host bacterial culture.

In an alternative group of embodiments, a method of preparing an hmw bacteriocin with a modified tail fiber as described herein is provided. The method may comprise preparing a nucleic acid molecule encoding a modified tail fiber protein by any means disclosed herein and expressing the nucleic acid molecule in a cell under conditions wherein an hmw bacteriocin is produced.

Compositions

In additional embodiments, an R-type pyocin or recombinant hmw bacteriocin of the disclosure may be present in a composition as described herein. In some embodiments, the composition may comprise an R-type pyocin and an additional agent for use in anti-Y. pestis therapy. Non-limiting examples of an additional agent include an antimicrobial, a bacteriophage, an antibiotic, an anti-fungal agent, an analgesic, and an anti-inflammatory agent. Non-limiting examples of an antibiotic include doxycycline, streptomycin, gentamycin, and ciprofloxacin.

The pyocin may optionally be isolated or purified from a naturally occurring source, such as, but not limited to, P. aeruginosa cells that produce it. As used herein, “isolated” or “purified” refer to the separation of a material from one or more other components normally found with the material. In many cases, the separation is from one or more proteins, lipids, carbohydrates, or nucleic acids normally found with the material. In only a few cases, the separation is to the level of high purity such that the other components are essentially absent. In other embodiments, the pyocin from a naturally occurring source, with the presence of one or more components normally found with the pyocin, is used in a composition of the disclosure.

A disclosed composition may also contain one or more carrier or excipient suitable for use in vivo or in vitro. With respect to in vivo embodiments, the composition may be formulated to be pharmaceutically acceptable so that the formulation may be used clinically or agriculturally. In some embodiments, the carrier or excipient is suitable for administration by an oral, topical, or inhalation route, such as to a human or other animal subject. In some cases, the formulation is suitable for application as an aerosol or dry inhalant.

In additional embodiments, a disclosed composition is formulated with a “pharmaceutically acceptable” excipient or carrier suitable for use with humans, animals, and/or plants without undue adverse side effects. Non-limiting examples of adverse side effects include toxicity, irritation, and/or allergic response. The excipient or carrier is typically one that is commensurate with a reasonable benefit/risk ratio. In many embodiments, the carrier or excipient is suitable for topical, oral, aerosol, inhaled, or systemic administration. Non-limiting pharmaceutically carriers include sterile aqueous or non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyloleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

With respect to in vitro embodiments, the carrier is one that does not negatively affect the Y. pestis inhibiting effects of an R-type pyocin or modified hmw bacteriocin. For example, a carrier that denatures the complex or sequesters it from binding Y. pestis would not be suitable for use in the practice of the disclosure.

Also provided are formulations comprising a stabilizing agent, wetting and emulsifying agent, salt for varying the osmotic pressure, or buffer for securing an adequate pH value may be included.

Modified Phage

In addition to modified hmw bacteriocins as described above, the disclosure includes the modified or recombinant phage that bind and kill Y. pestis. Such phage are modified to have the binding activity of an R2, R4, or R5 pyocin. In some embodiments, the phage have a tail fiber with an RBD from a pyocin tail fiber to facilitate binding between the phage and Y. pestis. As described above, the cognate chaperone corresponding to the RBD is often co-expressed with the modified tail fiber to promote or enable its proper folding and or attachment to the modified hmw bacteriocin. See FIG. 7. The binding of the modified phage tail fiber initiates the phage's infection and life cycle in Y. pestis. In some embodiments, the phage is a myoviridiae family member. Non-limiting examples include P2, P4, ΦA1122, and VHML phage. In other embodiments, the phage is a yersinophage. In further embodiments, the phage is one that is capable of replication in Y. pestis but lacks the ability to infect Y. pestis. In other embodiments, the phage is engineered via human intervention to express an R-type pyocin tail fiber protein RBD.

Methods of Use

In additional aspects, methods for the use of an R-type pyocin, a modified or recombinant hmw bacteriocin, or a modified or recombinant bacteriophage of the disclosure are provided. In some embodiments, a method of killing Y. pestis, optionally via loss of membrane potential or release of intracellular contents by compromising the integrity of the cytoplasmic membrane of a bacterium, is disclosed. The method may comprise contacting Y. pestis cells with an R-type pyocin, a modified or recombinant hmw bacteriocin, or a modified or recombinant bacteriophage as disclosed herein. Alternatively, the contact may be with a disclosed composition.

In additional embodiments, a method of preventing or inhibiting growth of Y. pestis as described herein may comprise treatment of a human patient or an animal subject. In some cases, the patient or subject is afflicted with, diagnosed as afflicted with, or suspected of being afflicted with, an infection by Y. pestis. Non-limiting examples of such a subject include animal (mammalian, reptilian, amphibian, and avian) species. Representative, and non-limiting, examples of mammalian species include humans; non-human primates; agriculturally relevant species such as cattle, pigs, goats, and sheep; rodents, such as mice and rats; mammals for companionship, display, or show, such as dogs, cats, guinea pigs, rabbits, and horses; and mammals for work, such as dogs and horses. Representative, and non-limiting, examples of avian species include chickens, ducks, geese, and birds for companionship or show, such as parrots and parakeets. An animal subject treated with an engineered bacteriocin of the disclosure may also be a quadruped, a biped, an aquatic animal, a vertebrate, or an invertebrate. In other embodiments, the animal subject is an arthropod, such as of the genus Xenopsylla as a non-limiting example. In some cases, the animal is a flea or other insect that is a carrier of Y. pestis.

In some embodiments, the subject to be treated is a human child or other young animal which has yet to reach maturity. Thus the disclosure includes the treatment of pediatric conditions comprising infection with Y. pestis.

The treatment of a patient or subject is typically treatment of an individual “in need of treatment”. The determination, or diagnosis, of the need for treatment may be made by a skilled person, such as a clinician, by use of art recognized means. In some embodiments, the subject is a patient or animal with a Y. pestis infection that is potentially life-threatening or that impairs health or shortens lifespan.

The methods of the disclosure may also be applied in an environment where Y. pestis growth is not desired or is considered to be harmful. Non-limiting examples include the sterilizing of environments, including medical settings and operating room facilities; as well as food preparation areas, including areas where raw meat or fish is handled. In further embodiments, an R-type pyocin or modified hmw bacteriocin of the disclosure may be used to treat a food product. The methods may also be used to sterilize heat sensitive objects, medical devices, and tissue implants, including transplant organs.

The methods can be used as a stand-alone therapy or as an adjunctive therapy, for targeting Y. pestis populations. Numerous antimicrobial agents (including antibiotics and chemotherapeutic agents) are known which would be useful in combination with these methods to treating bacteria-based conditions. In additional embodiments, a method to kill or inhibit the growth of Y. pestis in a biofilm form is provided. Such a method may comprise contacting a biofilm with an R-type pyocin, a modified or recombinant hmw bacteriocin, or a modified or recombinant bacteriophage as disclosed herein.

In further embodiments, and where Y. pestis is present on a surface of an inanimate object, the surface is contacted with an R-type pyocin, a modified or recombinant hmw bacteriocin, or a modified or recombinant bacteriophage that is applied to the surface. In many cases, the contact decontaminates said surface by killing the Y. pestis.

Additional embodiments include methods to deactivate a bioweapon comprising Y. pestis. The method may comprise contacting the bioweapon with an R-type pyocin, a modified or recombinant hmw bacteriocin, or a modified or recombinant bacteriophage as described herein. In some cases, the bioweapon is in the form of a solid, such as a powder, dust or other particulate form, which is mixed with an R-type pyocin, a modified or recombinant hmw bacteriocin, or a modified or recombinant bacteriophage of the disclosure, optionally in a liquid medium to facilitate dispersion.

Other methods of using the modified or recombinant phage include inoculating a subject to provide protection against Y. pestis. The method may include administering, to the subject, recombinant bacteria which express a modified or recombinant phage with the binding specificity as an R2, R4 or R5 pyocin. The production and so presence of the phage may be used to produce a protected state in the subject against infection or colonization by Y. pestis. In other embodiments, the recombinant bacteria express a modified or recombinant hmw bacteriocin as described herein to produce an analogous protected state.

In a further aspect, the disclosure includes a method to identify Y. pestis, optionally in the presence of one or more Yersinia species selected from Y. enterocolitica, Y. fredericksenii, and Y. pseudotuberculosis. The method may comprise contacting a sample containing one or more Yersinia species selected from Y. enterocolitica, Y. fredericksenii, and Y. pseudotuberculosis, with a detectably labeled R-type pyocin which binds Y. pestis to form a complex, and detecting the complex as an indicator of the presence of Y. pestis. Thus a method of detecting or identifying Y. pestis in a population of one or more of these additional species is provided. In some embodiments, the sample is suspected of containing Y. pestis. In additional embodiments, the sample is suspected of containing Y. pestis without knowledge regarding the possible presence of other Yersinia species.

Optionally, the method is used in combination with one or more nucleic acid or protein based assays to detect a Y. pestis specific sequence or polypeptide, respectively. Non-limiting examples include detection of Y. pestis specific genomic DNA or ribosomal RNA sequences or antibody-based detection of a Y. pestis specific polypeptide, such as Yop's.

In other embodiments, the method may comprise contacting a sample containing one or more Yersinia species selected from Y. enterocolitica, Y. fredericksenii, and Y. pseudotuberculosis, with an R-type pyocin, a modified or recombinant hmw bacteriocin, or a modified or recombinant bacteriophage as described herein, and detecting the release of one or more intercellular components from Y. pestis as an indicator of the presence of Y. pestis. In some cases, the detecting may be by a nucleic acid or protein based assay which detects a Y. pestis specific sequence or polypeptide, respectively, as described above. In some embodiments, the sample is suspected of containing Y. pestis. In additional embodiments, the sample is suspected of containing Y. pestis without knowledge regarding the possible presence of other Yersinia species.

As used herein, a “sample” or “test sample” refers to a sample isolated from an individual infected with, or suspected of being infected with, Y. pestis as well as environmental samples suspected of containing Y. pestis. Alternatively, the terms refer to samples known to contain Y. pestis for use as a control in the detection methods of the disclosure or for use in the disclosed detection methods to confirm the presence of, or quantify the amount of, Y. pestis cells. The sample may be collected by any appropriate means, including sampling of the outer skin or hair, as well as clothing, in cases of a animal or human subject. Sampling of air, paper, soil, or other solid objects may be used in cases of an environmental sample, such as that from a site suspected to contain Y. pestis. Samples of fleas or rodents may also be obtained and tested as described herein.

Medical samples also include sampling or swabbing of a subject's bodily surfaces, including, but not limited to, anal, nasal, otic and oral cavities or other squamous or mucosal tissue. Other sample forms include samples of water or food. A sample may also be a powder or granulated material suspected of containing Y. pestis. In some embodiments, a sample may be diluted with a sample diluent before being assayed. The diluent may be any suitable solvent as desired by the skilled person.

Where a method includes use of a detectably labeled material, the terms “label”, “detectably labeled” or “labeled with a detectable marker” refer to a composition capable of producing a detectable signal indicative of the presence of the labeled molecule. Suitable labels include radioisotopes, a dye, colloidal gold or a similarly detectable marker, nucleotide chromophores, enzymes, substrates, fluorescent molecules, chemiluminescent moieties, magnetic particles, bioluminescent moieties, and the like, including labels suitable for indirect detection, such as biotin. As such, a label is any composition detectable by spectroscopic, photochemical, biochemical, immunochemical, electrical, optical or chemical means. A label may be attached by use of a chemical linker. Exemplary labels are those that produce a visible signal that can be detected by visual inspection, such as with the unaided human eye.

Having now generally described the inventive subject matter, the same will be more readily understood through reference to the following examples which are provided by way of illustration, and are not intended to be limiting of the disclosure, unless specified.

### EXAMPLES

The following examples are offered to illustrate, but not to limit the claims below.

### Example 1

### R-Type Pyocins Kill Y. pestis

Lawns of P. aeruginosa strain 13s, see FIG. 5A, and Y. pestis KIM cells, see FIGS. 5B and C, were spot tested with isolated natural R1, R2, R3, R4, and R5 pyocins. The R2, R4 and R5 pyocins were observed to effectively kill Y. pestis KIM, as shown in FIGS. 5B and 5C. In addition, modified hmw bacteriocins compromising the substitution of an R2 RBD with that of an R2, R4 or R5 RBD, each expressed in trans, also killed Y. pestis KIM, as shown in FIG. 6D. Modified hmw bacteriocins compromising the substitution of an R2 RBD with that of an R1, R2, R3, R4 or R5 RBD, each expressed in trans, all killed P. aeruginosa strain 13s, see FIG. 6A, but not E. coli C, see FIG. 6C. The modified pyocin comprising the R5 RBD also killed an R5-sensitive, R2-resistant P. aeruginosa strain, see FIG. 6B, as well as Y. pestis KIM, see FIG. 6D.

### Example 2

### R2 Pyocin Mediated Killing of Y. pestis

An overnight culture of Y. pestis KIM cells was freshly diluted 1:10 or 1:50 in TSB in three identical 96-well plates. The cultures in the plates were incubated with R2 pyocin at 30° C. for 1, 3, or 6 hours. Portions of the cell/pyocin mixtures were then diluted and spotted on TSA plates, which were then incubated overnight at 30° C. Even one hour of incubation with pyocin at 30° C. was observed to produce cell killing. By determining the fractional survival of a known number of Y. pestis bacteria incubated with an unknown number of pyocin particles and deploying the method of Poisson, the number of pyocin particles added to each culture was determined. The number of pyocin particles is related to the fraction of bacterial survivors in a Poisson distribution, m=−1 nS, where m=the average number of lethal events/cell and S is the fraction of survivors. The total number of active pyocin particles/ml=m×cells/ml.

The results are shown below, where all numbers are given in pyocin particles per ml.

| | 1 hour | 3 hour | 6 hour |
|---|---|---|---|
| KIM 1:10 dilution | 2.1 × 10¹⁰ | 7.5 × 10¹⁰ | 2.2 × 10¹¹ |
| KIM 1:50 dilution | 1.0 × 10¹⁰ | 5.9 × 10¹⁰ | 2.4 × 10¹¹ |

### Example 3

### Y. pestis Specific Killing

The R2, R4 and R5 pyocins were observed to effectively kill Y. pestis KIM, but were not active against three other tested Yersinia species: namely Y. enterocolitica, Y. fredericksenii, and Y. pseudotuberculosis. Thus, R-2, R-4 and R-5 pyocins can distinguish Y. pestis from at least three other common Yersinia species (the negative data not shown).

### Example 4

### Relationship of R-Type Pyocin Binding Receptor to Yersiniophage Receptors

After mutagenesis with nitrosoguanidine, Yersinia pestis bacteria were selected for resistance to R-2 pyocin, and one isolated strain was further studied. The resistant strain was also resistant to R-4 and R-5 pyocins as well as to yersiniophage L413c. The R-2 resistant strain was still sensitive to yersiniophage ΦA1122. Thus R-2, R-4 and R-5 pyocins bind to a receptor on Y. pestis that is different than that responsible for binding ΦA1122 but related to (or overlapping with) the receptor that mediates binding by yersiniophage L-413c.

In a separate experiment and after mutagenesis of Y. pestis KIM, mutant bacteria were selected for resistance to yersiniophage L-413c and analyzed for sensitivity to R-2, R-4 and R-5 pyocins. Three phenotypes were found:
One phenotype was resistant to R-2, R-4, and R-5 pyocins and to the modified pyocins, R2-P2 and R2-L413, confirming at least some overlap of these pyocin receptors with the receptor for yersiniophage L-413c.
The second phenotype consisted of mutants still sensitive to R-2, R-4, and R-5 pyocins but resistant to the modified pyocins, R2-P2 and R2-L413. These mutants may have resulted from mutations in Y. pestis genes encoding receptors not overlapping with the receptor for the natural R-type pyocins.
The third phenotype was still sensitive to R-2, R-4 and R-5 pyocins and to the modified pyocins, R2-P2 and R2-L413. These latter mutants may have resulted from mutations in Y. pestis genes encoding one or more cellular functions necessary for phage infection-replication but not required for killing by pyocins.

### REFERENCES

Anisimov, A B and K K Amoako. 2006. Treatment of plague: promising alternatives to antibiotics. J Med Microbiol. 2006 55:1461-75.
Bertani L E, and E W Six. 1988. The P2-like phages and their parasite, P4. In R. Calendar (ed.), The Bacteriophages, vol. 2. Plenum Publishing Corp., New York. pp 73-143
Birmingham V A, P A Pattee. 1981. Genetic Transformation in Staphylococcus aureus: Isolation and Characterization of a Competence-Conferring Factor from Bacteriophage 80α Lysates. Journal of Bacteriology 148:301-307
Blackwell C C, F P Winstanley, W A Telfer-Brunton. 1982. Sensitivity of thermophilic campylobacters to R-type pyocins of Pseudomonas aeruginosa. J. Med Microbiology. 15:247-51
Bradley D E. 1967. Ultrastructure of bacteriophage and bacteriocins. Bacteriol Rev. 31:230-314.
Burda M R, and S. Miller. 1999. Folding of coliphage T4 short tail fiber in vitro. Analysing the role of a bacteriophage-encoded chaperone. Eur J Biochem. 1999 October; 265(2):771-8.
Coetzee H L, H C De Klerk, J N Coetzee, J A Smit. 1968. Bacteriophage-tail-like particles associated with intra-species killing of Proteus vulgaris. J Gen Virol. 2:29-36.
Daw M A, and F R Fraliner 1996. Bacteriocins: nature, function and structure. Micron. 27:467-79.
Dyke J, Berk R S. Growth inhibition and pyocin receptor properties of endotoxin from Pseudomonas aeruginosa. Proc Soc Exp Biol Med. 1974; 145:1405-1408.
Epidemiologic Fingerprinting of Pseudomonas aeruginosa by the Production of and Sensitivity to Pyocin and Bacteriophage. Applied Microbiol. 18:760-765
Haas H, T Sacks, N Saltz. 1974. Protective Effect of Pyocin Against Lethal Pseudomonas aeruginosa Infections in Mice. J. of Infectious Diseases. 129:470-472
Haggard-Ljungquist E, C Halling, R Calendar. 1992. DNA Sequences of the Tail Fiber Genes of Bacteriophage P2: Evidence for Horizontal Transfer of Tail Fiber Genes Among Unrelated Bacteriophages. Journal of Bacteriology. 174:1462-1477.
Hashemolhosseini S, Montag D, Kramer L, and U Henning. 1994. U.Determinants of receptor specificity of coliphages of the T4 family. A chaperone alters the host range. J Mol Biol. 241:524-33.
Ishii S, Y. Nishi, and F Egami. 1965. The fine structure of a pyocin. J. Mol. Biol. 13:428-431
Iijima M. 1978. Mode of Action of Pyocin R1. J. Biochem (Tokyo) 83:395-402.
Jabrane A, Sabri A, Compere P, Jacques P, Vandenberghe I, Van Beeumen J, and P Thonart. 2002. Characterization of serracin P, a phage-tail-like bacteriocin, and its activity against Erwinia amylovora, the fire blight pathogen. Appl Environ Microbiol. 68:5704-10.
Jacob F. 1954. Biosynthèse induite et mode d'action d'une pyocin, antibiotique de Pseudomonas pyocyanea. Annals Inst. Pasteur. 86:149-60
Kageyama M, F Egami. 1962. On the purification and some properties of a pyocin, a bacteriocin produced by Pseudomonas aeruginosa. Life Sciences 9: 471-6
Kageyama M. 1964. Studies of a pyocin I. Physical and chemical properties. J. Biochem. 55:49-53
Kageyama M, K Ikeda, and F Egami. 1964. Studies of a pyocin. III. Biological properties of the pyocin. J. Biochem. 55:59-64.
Kageyama M. 1975. Bacteriocins and Bacteriophages in Pseudomonas aeruginosa, in Microbial Drug Resistance. Mitsuhashi, T, and Hashimoto, H (eds). University of Tokyo Press, Tokyo. pp. 291-305
Kageyama M, Shimomiya T, Aihara Y, Kobayashi M. 1979 Characterization of a bacteriophage related to R-type pyocins. J Virol. 32:951-957.
Kahn M L, R G Ziermann, D W Deho, M Ow, G Sunshine, R Calendar. 1991. Bacteriophage P2 and P4. Methods Enzymol. 204:264-280
Kingsbury, D T. 1966 Bacteriocin production by strains of Neisseria meningitidis. J Bacteriol. 91:1696-9.
Kumazaki T, Y. Shimizu, S I Ishii. 1982. Isolation and Characterization of Pyocin R1 Fibers. J. Biochemistry. 91:825-35
Lee F K, Dudas K C, Hanson J A, Nelson M B, LoVerde P T, Apicella M A. 1999 The R-type pyocin of Pseudomonas aeruginosa C is a bacteriophage tail-like particle that contains single-stranded DNA. Infect Immun. 67(2):717-25.
Matsui H, Sano Y, Ishihara H, Shinomiya T. 1993 Regulation of pyocin genes in Pseudomonas aeruginosa by positive (prtN) and negative (prtR) regulatory genes. J Bacteriol. 175:1257-1263.
Merrikin D J, C S Terry. 1972. Use of Pyocin 78-C2 in the Treatment of Pseudomonas aeruginosa Infection in Mice. Applied Microbiology, 23:164-165
Morse S A, P Vaughan, D Johnson, B H Iglewski. 1976. Inhibition of Neisseria gonorrhoeae by a Bacteriocin from Pseudomonas aeruginosa. Antimicrobial Agents and Chemotherapy 10:354-362
Morse S A, B Y Jones, P G Lysko. 1980. Pyocin Inhibition of Neisseria gonorrhoeae: Mechanism of Action. Antimicrobial Agents and Chemotherapy. 18:416-423
Nakayama K, K Shigehiko, M Ohnishi, Y Teryaki, T Hayashi. 1999. The Complete Nucleotide Sequence of ΦCTX, a cytotoxic-converting phage of Pseudomonas aeruginosa: implications for phage evolution and horizontal gene transfer via bacteriophages. Molecular Microbiology 31:399-419
Nakayama K, K Takashima, H Ishihara, T Shinomiya, M Kageyama, S Kanaya M Ohnishi, T Murata, H Mori, T Hayashi. 2000. The R-type pyocin of Pseudomonas aeruginosa is related to P2 phage, and the F-type is related to lambda phage. Molecular Microbiology. 38:213-231
Qu Y, Hyman P, Harrah T, and E. Goldberg. 2004. In vivo bypass of chaperone by extended coiled-coil motif in T4 tail fiber. J Bacteriol. 186:8363-9.
Shimizu Y, T Kamazaki, S I Ishii. 1982. Specific Cleavage at Fibers of a Bacteriophage-Tail-Like Bacteriocin, Pyocin R1 by Successive Treatment with Organomercurial Compounds and Trypsin. J Virology 44:692-695
Shinomiya T, S Shiga. 1979. Bactericidal Activity of the Tail of Pseudomonas aeruginosa Bacteriophage PS17. J of Virology 32:958-967
Shinomiya T, S Shiga, M Kageyama. 1983a. Genetic determinant of pyocin R2 in Pseudomonas aeruginosa PAO. I. Localization of the pyocin R2 gene cluster between the trpCD and trpE genes. Mol Gen Genet. 189:375-38
Shinomiya T, S Shiga, A Kikuchi, M Kageyama. 1983b. Genetic determinant of pyocin R2 in Pseudomonas aeruginosa PAO. II. Physical characterization of pyocin R2 genes using R-prime plasmids constructed from R68.45. Mol Gen Genet. 189:382-389
Shinomiya T. 1984. Phenotypic Mixing of Pyocin R2 and Bacteriophage PS17 in Pseudomonas aeruginosa PAO. J. Virology. 49:310-314
Shinomiya T & S Ina. 1989. Genetic Comparison of Bacteriophage PS17 and Pseudomonas aeruginosa R-Type Pyocin. J. Bacteriology 171:2287-2292
Strauch E, H Kaspar, C Schaudinn, P Dersch, K Madela, C Gewinner, S Hertwig, J O Wecke, B Appel. 2001. Characterization of Enterocoliticin, a Phage Tail-Like Bacteriocin, and Its Effect on Pathogenic Yersinia enterocolitica Strains. Applied and Environmental Microbiology. 67:5634-5642
Thompson N E, P A Pattee. 1981. Genetic transformation in Staphylococcus aureus: demonstration of a competence-conferring factor of bacteriophage origin in bacteriophage 80a lysates. J. Bacteriol. 148:294-300
Uratani Y, T Hoshino. 1984. Pyocin R1 Inhibits Active Transport in Pseudomonas aeruginosa and Depolarizes Membrane Potential. Journal of Bacteriology. 157:632-636
Zink R, M J Loessner and S Scherer. 1995. Characterization of cryptic prophages (monocins) in Listeria and sequence analysis of a holin/endolysin gene. Microbiology. 141:2577-2584

All references cited herein are hereby incorporated by reference in their entireties, whether previously specifically incorporated or not. As used herein, the terms “a”, “an”, and “any” are each intended to include both the singular and plural forms.

Having now fully described the disclosed subject matter, it will be appreciated by those skilled in the art that the same can be performed within a wide range of equivalent parameters, concentrations, and conditions without departing from the spirit and scope of the disclosure and without undue experimentation. While this disclosure has been described in connection with specific embodiments thereof, it will be understood that it is capable of further modifications. This application is intended to cover any variations, uses, or adaptations of the subject matter following, in general, the principles of the disclosure and including such departures from the disclosure as come within known or customary practice within the art to which the subject matter pertains and as may be applied to the essential features hereinbefore set forth.

What is claimed is:

1. A method of inhibiting growth of Y. pestis, said method comprising contacting said Y. pestis with an isolated R-type pyocin which binds thereto.

2. The method of claim 1, wherein contacting kills said Y. pestis.

3. The method of claim 1, wherein said pyocin is an R2, R4, or R5 type pyocin.

4. The method of claim 1, further comprising contacting said Y. pestis with an antibiotic.

5. The method of claim 1, wherein said contacting occurs in vivo or in vitro.

6. The method of claim 5, wherein said contacting occurs in vivo and bacteria are present in a human patient or an animal subject.

7. The method of claim 6, wherein said animal subject is an arthropod of the genus Xenopsylla or a flea.

8. The method of claim 5, wherein said contacting is in vitro; said Y. pestis is present on a surface of an inanimate object; and said contacting decontaminates said surface by killing the Y. pestis.

9. The method of claim 6, wherein said contacting comprises administering said pyocin to said patient or subject.

10. The method of claim 9, wherein said pyocin is in a composition comprising a pharmaceutically acceptable excipient.

11. The method of claim 10, wherein said composition is formulated for administration by an oral, topical, or inhalation route.

12. A method of inhibiting growth of Y. pestis, said method comprising contacting Y. pestis with a recombinant bacteriophage comprising a receptor binding domain from an R-type pyocin.

13. A method of inoculating a subject, said method comprising administering to said subject, recombinant bacteria which express a recombinant bacteriophage or hmw bacteriocin comprising a receptor binding domain (RBD) from an R-type pyocin.

14. The method of claim 13, wherein said RBD is that of an R2, R4, or R5 type pyocin.

15. The method of claim 14, wherein said bacteriophage is a myoviridiae family member, optionally selected from P2, P4, ΦA1122, ΦL-413c and VHML.

16. The method of claim 15, wherein said subject is a human being, a mammalian animal, or an arthropod.

17. The method of claim 16, wherein said animal subject is an arthropod of the genus Xenopsylla or a flea.

18. A method of deactivating a bioweapon comprising Y. pestis, said method comprising contacting said bioweapon with an R-type pyocin which compromises the integrity of a Y. pestis cytoplasmic membrane.

19. A composition comprising an R-type pyocin and a Yersinia inhibiting antibiotic.

20. The composition of claim 19, wherein said pyocin is an isolated or recombinant R-type pyocin.

21. The composition of claim 20, wherein said R-type pyocin is an R2, R4, or R5 type pyocin.

22. The composition of claim 21, wherein said antibiotic is doxycycline, streptomycin, gentamicin, or ciprofloxacin.

23. A method of detecting the presence of Y. pestis in a sample suspected of containing Y. pestis, said method comprising
contacting a sample with a detectably labeled R-type pyocin which binds Y. pestis to form a complex, and
detecting said complex as an indicator of the presence of Y. pestis.

24. The method of claim 23, wherein the sample is further suspected to contain one or more Yersinia species.

25. The method of claim 24, wherein the Yersinia species is Y. enterocolitica, Y. fredericksenii, and/or Y. pseudotuberculosis.

26. A method of detecting the presence of Y. pestis in a sample suspected of containing Y. pestis, said method comprising
contacting a sample, with an R-type pyocin which compromises the integrity of the cytoplasmic membrane of Y. pestis, and
detecting the release of one or more intercellular components from said Y. pestis as an indicator of the presence of Y. pestis.

27. The method of claim 26, wherein the sample is further suspected to contain one or more Yersinia species.

28. The method of claim 27, wherein the Yersinia species is Y. enterocolitica, Y. fredericksenii, and/or Y. pseudotuberculosis.
